(19) United States
(12) Patent Application Publication
(10) Pub. No.: US 2009/0054878 A1
(43) Pub. Date: Feb. 26, 2009

(54) Introduction device for a medical instrument

(76) Inventors: John S. Geis, Bad Zwischenhahn (DE); Michael Braun, Backnang (DE)

Correspondence Address: Mark J. Pandiscio;Pandiscio & Pandiscio, P.C., 470 Totten Pond Road, Waltham, MA, 02451-1914, US

(21) Appl. No.: 11/895,249

(22) Filed: Aug. 23, 2007

(51) Int. Cl. A61B 17/00

(52) U.S. Cl. 606/1

(57) ABSTRACT

The invention relates to a device for introducing a medical instrument into a closed container (2). The device is characterized in that it has a penetration means (11) for penetration of a container wall and a guiding unit (12) is adjacent to the penetration means (11), wherein the penetration means (11) and the guiding unit (12) have a common passage (10) for the introduction of a medical instrument (3) and the passage (10) has at least temporarily a narrowing.

### FIELD OF THE INVENTION

The present invention relates to an introduction device for a medical instrument.

### BACKGROUND OF THE INVENTION

In interventional medicine, such as for example interventional cardiology or radiology, it may be necessary to deliver substances via catheter or other elongated medical instruments to a location which is to be treated within the body of a patient. For this purpose, the substance has to be applied onto the medical instrument or into the medical instrument. Therefore, the medical instruments may possess a suitable surface structure or coating which receives the substance or which can be activated by the substance, respectively. Furthermore, it may be necessary, for example for cleaning purposes, to insert a medical instrument into a solution or another substance or to bring it into contact therewith, respectively.

It is known to immerse medical instruments in a substance which is provided in an open container. This kind of introducing or applying of the substance into or onto the instrument or the contacting of the medical instrument with a substance, respectively, however, has the disadvantage that in order to facilitate the immersing process, the opening of the container has to be chosen to be large. This process is thus disadvantageous with substances which are volatile or react with air in any other way.

Furthermore, applying or introducing of volatile or otherwise reactive substances into the instrument in a sterile room and subsequent hermetic packaging of the instrument, for example by means of a protective sheath, is only possible under certain conditions. Firstly, the surgeon has to remove a protective sheath during the treatment of the patient, in particular immediately before the intervention, in order to avoid volatilization or other reaction of the substance with ambient air. Secondly, such an instrument can only be used once for introducing of the substance into the body.

Also the usage of a container which is closed with a lid, wherein the lid is removed on-site and wherein the lid can be placed back after the usage of the substance, is not an alternative for the surgeon, as the opening and closing of the container is cumbersome but a complete sealing of the container is necessary, in order to avoid volatilization of the substance from the container.

The problem underlying the present invention is thus to provide a device which allows a surgeon to easily and potentially on-site bring the a medical instrument at least partially in contact with a substance in a container and nevertheless avoids volatilization, leaking and other reactions of the substance with the ambient air.

### SUMMARY OF THE INVENTION

This problem is solved according to the invention by a device for introducing a medical instrument into a closed container. The device is characterized in that it has a penetration means for penetration of a container wall and in that a guiding unit is adjacent to the penetration means, wherein the penetration means and the guiding unit have a common passage for the introduction of a medical instrument, and in that the passage at least temporarily has a narrowing.

For the purpose of the invention, containers with a hermetically sealed interior space are referred to as closed containers. These containers preferably have a cap made of a material which is different from the material of the remaining container walls which for example can be made of glass. It is in particular preferable if the cap is made of a flexible, in particular elastic material, which can be penetrated by a pointy object.

The device according to the present invention is provided with a penetration means for penetrating the at least one container wall, in particular the cap of the container. The penetration means is adjacent to the guiding unit. After inserting the penetration means into the container wall, in particular the container cap, the guiding unit is accessible for the user from the outside. A medical instrument can at least partially be inserted through the passage extending through the two components, namely the penetration means and the guiding unit, from one end of the guiding unit through the guiding unit and through the penetration means into the inner space of the container. The medical instrument is preferably an elongated instrument, in particular a wire or a catheter.

By means of the narrowing, which is provided in the passage at least at the inner diameter over its length, various advantageous functions can be realized with the device according to the invention. Firstly, the narrowing from the first end of the guiding unit to the narrowest point of the passage can assure guidance. This means that an opening with a small diameter does not have to be threaded cumbersomely with the medical instrument which is to be introduced into the interior space of the container. Furthermore, the narrowing can mostly avoid leaking of substance from the container and a reaction of the substance with the ambient air.

According to one embodiment, the narrowing of the passage is formed by the geometry of the guiding unit and is permanently present. For example, a first end of the guiding unit may be formed as a funnel. The diameter of the passage at the narrowest position of the funnel in this embodiment may correspond to the inner diameter of the penetration means. The funnel shape allows for choosing a sufficiently large diameter of the passage at the introduction end of the guiding unit via which the medical instrument is being inserted, which facilitates the introduction of the medical instrument by the user. Simultaneously, it is possible to choose the diameter of the narrowing such that it basically corresponds to the diameter of the medical instrument. Instruments which are used for interventional treatments in general have a very small diameter. Therefore also the diameter of the funnel may be chosen to be small and thus a coming out of the substance from the container can essentially be avoided.

Alternatively or additionally, the narrowing may temporarily be created by a valve, in particular a check valve, which is provided in the guiding unit.

Thereby, the leaking and/or the coming in contact of the substance with the ambient air can reliably be avoided. Particularly valves operable by pressure may be chosen as check valves. Such a valve is preferably provided in the area of the guiding unit or the guiding unit is being formed by the valve, respectively. For example a clamp valve may be considered as a valve which is operated by pressure. Particularly preferably, a valve which can be operated with one hand is being used. If necessary, the surgeon can thereby introduce the medical instrument easily into the container without having to lay down the instrument beforehand.

The penetration means according to the invention may at least partially be surrounded by a stabilizing device. This stabilizing device serves for stabilizing the introduction device of the invention in a position relative to the container. Thereby, tilting of the introduction device in the introduced state relative to the container can be avoided and thus the risk of leaking of the substance can be avoided. The stabilizing device may be a simple plate which supports the guiding unit on the surface of the container. Preferably, the stabilization device, however, is formed as a sleeve, which is covered by a plate on one side. Thereby, the stabilizing device receives the shape of a pot. Within this pot the penetration means is provided. Thereby, the stabilizing device can serve as protection, in a state where the introduction device has not been introduced into the container. On the one hand, a deformation of the penetration means can be avoided by the stabilizing device which also surrounds the penetration means over its length. On the other hand there is no risk of injuring the user in this state. The length of the stabilizing means, in particular of the sleeve, is preferably chosen such that it is larger then the length of the part of the penetration means which is surrounded by the sleeve. In particular, a ratio of 3 to 2 for the length of the sleeve to the length of the part of the penetration means which is surrounded by the sleeve is preferred. The length of the sleeve can be chosen in particular dependent on the height of the container, the container wall of which is to be penetrated. Preferably, the sleeve in the introduced state of the penetration means into the container covers 2/3 of the height of the container. Thereby the stability of the connection between the introduction device and the container can be increased.

The penetration means according to one embodiment basically has a pipe-shape and has a tip on one end. Insofar, the penetration means may basically have the shape of a syringe tip.

The guiding unit and the stabilizing device are preferably designed integrally. Hereby, the manufacturing of the introduction device of the invention can be simplified and the handling thereof can be facilitated. In this embodiment, the assembly of several components of the introduction device is not necessary, neither during the manufacturing process or on-site. It is particularly preferable if the introduction device is manufactured by an injection molding process. The penetration means may therein be provided integrally with the guiding unit or the stabilizing device.

According to a further embodiment, the guiding unit is partially formed by the penetration means. Herein, for example a lower section of the guiding unit can be formed by the penetration means and a valve may be arranged on top thereof. In this case, the narrowing of the passage will be formed in the area of the valve.

The valve, which according to the present invention may serve for temporarily creating a narrowing, may for example comprise a swivel arm and a spring, wherein the spring acts on the swivel arm. The valve may be designed to either narrow the passage, when the spring is in a relaxed state or when the spring is in the tense state. In the latter case, a locking means for locking the swivel arm may be provided on the introduction device, whereby the valve can be kept in a closed position, without necessitating the user operating it.

Most preferably, the temporary narrowing of the passage leads to a temporary closing thereof.

The diameter of the passage is preferably chosen such that with a permanent narrowing at this position the diameter corresponds to the diameter of the medical instrument. With the embodiment, wherein the narrowing is created temporarily, in contrast, the passage is preferably designed in an un-narrowed state such that its diameter over the major part of its length generally corresponds to the outer diameter of the medical instrument.

Features and advantages, which are described with reference to the device according to the invention, where appropriate, also apply correspondingly to the process of the invention.

### BRIEF DESCRIPTION OF THE DRAWINGS

The invention will be explained hereinafter with reference to the attached drawings which refer to possible embodiments of the invention.

FIG. 1: shows a schematic depiction of a first embodiment of the device according to the invention;

FIG. 2: shows a schematic depiction of a further embodiment of the device according to the invention;

FIG. 3: shows a schematic depiction of a further embodiment of the device according to the invention; and

FIG. 4: shows a schematic depiction of a device according to FIG. 2 in a state connected to a container.

The depictions are not to scale.

### DETAILED DESCRIPTION OF THE PREFERRED EMBODIMENT

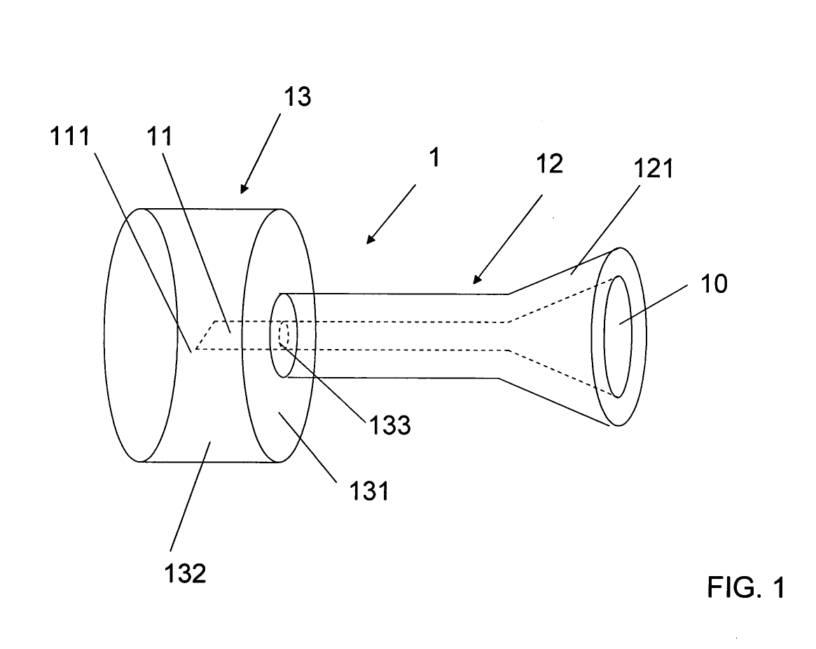

FIG. 1 is a schematic depiction of a first embodiment of the introduction device 1 according to the invention. In the depicted embodiment the introduction device 1 consists of a penetration means 11, a guiding unit 12 and a stabilization device 13.

The stabilization device 13 has the shape of a lid with a cover plate 131 and a cylinder 132 connected thereto, wherein the cylinder forms the sidewall of the lid. An aperture 133 is provided in the cover plate 131 and the penetration means 11 is in connection with the aperture 133. In the depicted embodiment, the penetration means 11 is realized as a pipe piece, the length of which varies over the circumference thereof such that at the bottom end of the penetration means 11 a tip 111 is formed.

The height or length, respectively, of the stabilization device 13 is preferably chosen such that it is larger that the length of the penetration means 11. Thereby, the penetration means 11 is protected.

The guiding unit 12 is provided at the upper side of the cover plate 131. The guiding unit 12 is preferably formed as one piece with the stabilization device 13, in particular with the cover plate 131.

In the depicted embodiment, the guiding unit 12 has a funnel shape, wherein the enlarged part of the funnel 121 is on the end of the guiding unit 12, which is opposite to the stabilizing unit 13 and to the penetration means 11. The inner diameter of the narrowed part of the funnel corresponds to the inner diameter of the penetration means 11. Thereby no step is formed at the transition between the penetration means 11 and the guiding unit 12. According to the present invention, it is, however, also possible to choose the inner diameter of the lower part of the funnel to be smaller than the inner diameter of the penetration means 11. Also in this embodiment the transition from the funnel to the penetration means 11 coming from the funnel is without a step.

A medical instrument can be guided through the passage 10 which is formed by the inner diameter of the guiding unit 12 and the inner diameter of the penetration means 11. This medical instrument may for example be a wire, a catheter or another longitudinally extending instrument.

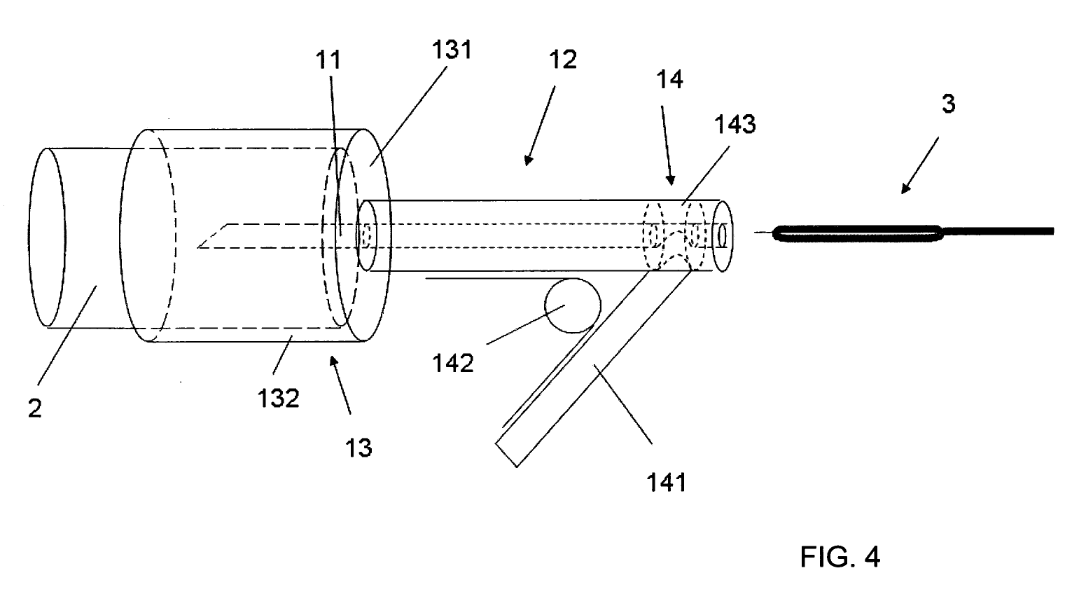

If the introduction unit 1 is placed onto a container 2 (FIG. 4), the lower end of the penetration means 11, which is realized as a tip 111, will come in contact with a wall, in particular a lid of the container 2 and in case of an appropriate choice of the material of the container 2 may penetrate at this area through the wall. Thereby, the interior of the container 2, which is essentially closed, will be accessible by a medical instrument through the passage 10 of the introduction device 1.

It is also possible that the penetration means 11 is formed as one piece with at least a part of the guiding unit 12. In this case, the penetration means 11 may for example form a unity with the lower part of the funnel and the conical section of the funnel may be located on top. Furthermore, it is possible, that the penetration means 11 and the entire guiding unit 12 is formed as a common unit, wherein, where applicable, also the stabilization unit 13 may be formed integrally with the other elements 11, 12.

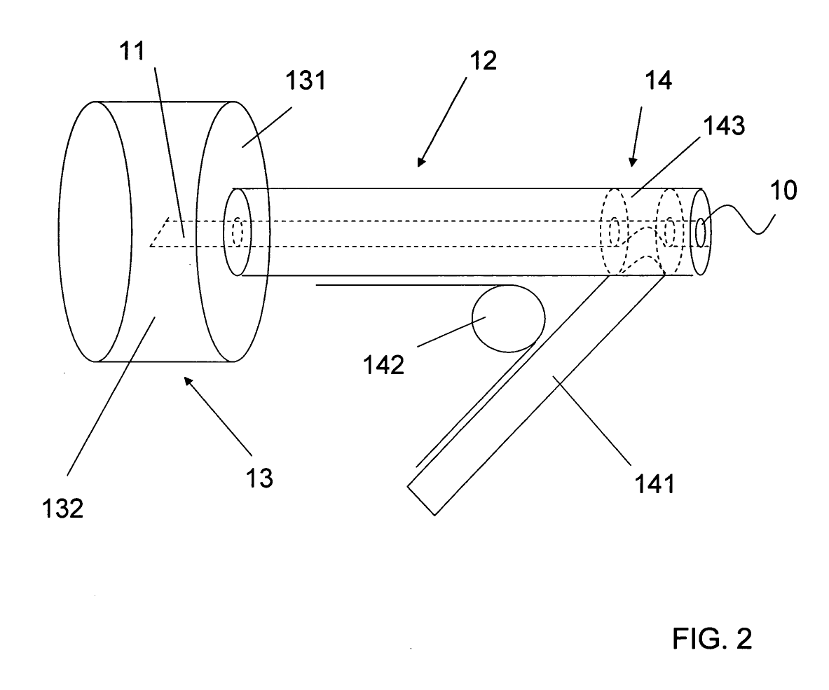

In FIG. 2 a further embodiment of the introduction device 1 according to the invention is shown. In the lower section, i.e. in the section of the penetration means 11 and the stabilization device 13, this introduction unit 1 has the same design as the embodiment shown in FIG. 1. The components of this section and their function as well as their advantages described with reference to FIG. 1 will therefore not be described again. The same components are marked with corresponding reference numbers.

In the embodiment depicted in FIG. 2, however, a valve 14, in particular a check valve, is provided in the guiding unit 12. In this embodiment the narrowing is created in the passage 10 of the introduction device, when the valve is in its resting position. If the valve is, however, actuated, the passage 10 will resume a continuous cylindrical shape. In this state a medical instrument can be guided through the passage 10. With the valve 14 being in a resting position the passage 10 will be closed and coming out of substances of a container 2 which is connected to the introduction device 1 can be avoided.

In the depicted embodiment, the valve 14 comprises a swivel arm 141, which is connected to the tubular part of the guiding unit 12 and which is in particular arranged laterally thereon. In the tube a sealing means 143 is provided in the area of the connection point at which the swivel arm 141 is connected pivotable to the tubular area. The sealing means 143 has an annular shape. If the swivel arm 141 is released, a spring 142 which is provided on the outside of the tube can push the swivel arm 141 in a position, such that it acts against the sealing means 143. The sealing means 143 is made of elastic material and can be deformed by the influence of the swivel arm 141. In particular, the ring will be deformed such that it closes the passage 10 within the introduction device 1.

Alternatively, it is also possible to design the check valve 14 such that it acts in the opposite direction, namely that it closes the passage 10 when the swivel arm 141 is pushed against the tube. This may be realized by means of a sling, which contracts upon actuation of the swivel arm 141.

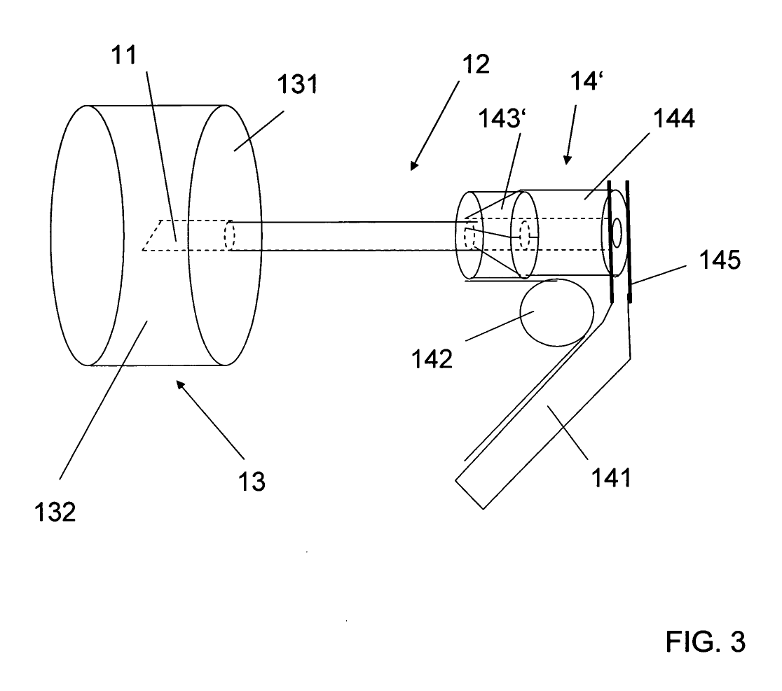

In FIG. 3 a further embodiment of the introduction device 1 according to the invention is shown. In this embodiment, the guiding unit 12 is formed at its lower part by the penetration means 11. A valve 14′ is placed on top thereof and forms the upper part of the guiding unit 12. The valve shown in FIG. 3 has a different design compared to the valve shown in FIG. 2. Within valve 14′ a sealing means 143′ is provided, which has a conical shape and consists of two halves. In the depicted embodiment the sealing means 143′ is arranged such that the cone tip is facing the penetration means 11. In the sealing means 143′ a passage is provided, which corresponds to the passage in the penetration means 11. On the side of the cone facing away from the penetration means 11, a piston 144 is provided and through which also a passage extends. The cone 143′ is made of elastic material, whereas the piston 144 consists of a rigid material.

At the swivel arm 141 two push arms 145 are provided in the depicted embodiment. These push arms 145 extend along the surface of the piston 144. By means of the push arms 145 pressure can be applied onto the piston via the swivel arm 141. The pressure on the piston 144 in turn applies pressure onto the sealing means 143′. The sealing means 143′ will deform due to the pressure acting on it and will thus close the passage 10 through the guiding unit 12.

In the depicted embodiment the valve 14′ is closed in the resting position, i.e. with the spring 142 being relaxed. If, however, the swivel arm 141 is moved towards the guiding unit 12, the pressure will be released from the sealing means 143′ and it will open the passage 10. In this state the user can guide a medical instrument through the passage 10.

Finally, FIG. 4 shows the embodiment of the introduction device 1 according to FIG. 2 in the state, in which it is connected to, in particular plugged into, a container 2, wherein a substance may be provided.

In this state the penetration means 11 extends through the upper side of the container 2, which may for example be made of an elastic material.

In this state, when the valve 14 is actuated by pivoting of the swivel arm 141 towards the guiding unit 12, a medical instrument 3 can thus be introduced into the interior of the container 2 via the introduction device 1 und may there be coated or soaked with a substance. After the removal of the medical instrument, the valve 14 can be brought into its resting position by releasing the swivel arm 141, whereby the passage closes again. In this state, the container 2 can thus also be laid down without the substance coming out of the container. This is specifically advantageous during a surgical, in particular a minimal invasive intervention, as the surgeon does not have to pay specific attention to the container 2 but can concentrate on the surgery. In case further substance is needed, the surgeon can, however, use the container 2 again in the described way.

Also in the embodiment according to FIG. 2, the penetration means 11 and the lower part of the guiding unit 12 can be integral with each other, whereby the valve 14 is positioned on top of the upper end of the penetration means and forms the upper part of the guiding unit 12.

Particularly preferably, also the embodiments shown in FIGS. 2 and 3 have a widened diameter in their upper part. Herewith, a facilitated introduction of the medical instrument into the introduction device 1 becomes possible. The widening of the diameter can occur due to the provisioning of a conical area at the upper end of the guiding unit 12 or due a widening by steps.

The invention is not limited to the depicted embodiments.

The introduction device can be made of various materials. The penetration means is preferably made of a metal or plastic with high strength. In the latter case the entire introduction device can be made as one piece of the same material. This allows for minimizing the production costs of the device of the invention. It is in particular possible that the entire device is produced in an injection molding process. Also with a device, wherein the penetration means is made of metal, the guiding unit and the stabilization device, if provided, can be made by injection molding.

The dimensions of the individual components of the device can be chosen according to the respective needs. In particular, for the usage for medical instruments, which are used in minimal invasive surgery, in particular the inner diameter of the passage will be chosen to be small and may for example be in the range of 3 to 8 French (1 mm to 2.66 mm). With these dimensions, on the one hand the introduction of a catheter or wire into a container to be connected with the introduction device can be assured. On the other hand, however, in the embodiment, wherein the device is present without a valve, a leakage or coming out of the substance from the container can be avoided.

What is claimed is:

1. Device for introducing a medical instrument into a closed container (2), the device comprising a penetration means (11) for penetration of a container wall and a guiding unit (12) adjacent to the penetration means (11), wherein the penetration means (11) and the guiding unit (12) have a common passage (10) for the introduction of a medical instrument (3) and the passage (10) exhibits at least temporarily a narrowing.

2. Device according to claim 1, wherein the narrowing of the passage (10) is formed by the geometry of the guiding unit (12) and is permanent.

3. Device according to claim 1, wherein the narrowing is generated temporarily, wherein in particular a valve (14, 14′) is provided in the guiding unit (12).

4. Device according to claim 1, wherein the penetration means (11) is at least partially surrounded by a stabilization device (13).

5. Device according to claim 1, wherein the penetration means (11) essentially has a pipe shape and a tip (111).

6. Device according to claim 4, wherein the length of the stabilization device (13) corresponds at least to the length of the penetration means (11) in the area, where it is surrounded by the stabilization device (13).

7. Device according to claim 4, wherein the guiding unit (12) and the stabilization device (13) are integrally formed.

8. Device according to claim 1, wherein the guiding unit (12) is at least partially formed by the penetration means (11).

9. Device according to claim 2 wherein the passage (10) in the area of the permanent narrowing, where the narrowing is created, corresponds to the outer diameter of the medical instrument (3) which is to be inserted into the introduction device (1).

10. Device according to claim 1, wherein the device is manufactured by an injection molding process.

11. Device according to claim 3, wherein the passage (10) in the area of the temporarily formed narrowing in the area, where the narrowing is created, corresponds to the outer diameter of the medical instrument (3) which is to be inserted into the introduction device (1).
